(19) United States
(12) Patent Application Publication
(10) Pub. No.: US 2002/0188123 A1
(43) Pub. Date: Dec. 12, 2002

(54) Polydentate asymmetric ligands based on binaphthyl

(75) Inventors: Axel Monsees, Frankfurt (DE); Carsten A. Schneider, Frankfurt (DE); Thomas Riermeier, Floersheim (DE); Uwe Dingerdissen, Seeheim-Jugenheim (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 10/136,323

(22) Filed: May 2, 2002

(30) Foreign Application Priority Data
May 9, 2001 (DE) 101 22 539.3

(51) Int. Cl. C07D 277/28; C07D 237/02; C07D 263/30

(52) U.S. Cl. 544/224; 544/242; 544/336; 546/329; 548/205; 548/235; 548/343.5; 548/444; 548/577; 564/271

(57) ABSTRACT

Novel polydentate asymmetric nitrogen-oxygen-containing binaphthyl derivatives of the formulae (I) and (II), and metal complexes of these compounds may be used as catalysts for enantioselective transformations.

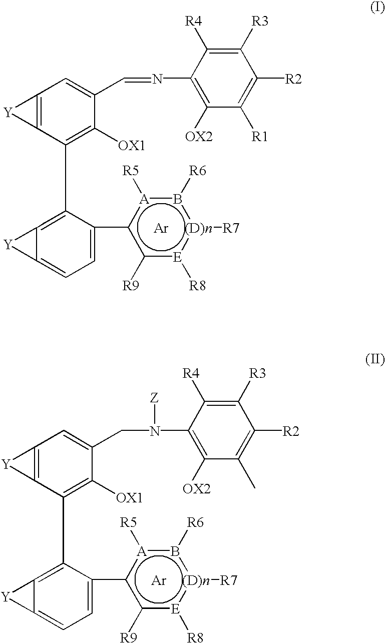

### BACKGROUND OF THE INVENTION

1. Field of the Invention

The present invention relates to novel polydentate asymmetric binaphthyl derivatives and methods of synthesizing these derivatives, and also complexes of these derivatives with metals and enantioselective transformations catalyzed by these complexes.

2. Discussion of the Background

Enantiomerically enriched chiral, polydentate ligands are used in asymmetric synthesis or asymmetric catalysis. In asymmetric catalysis, it is important that the electronic properties and the stereochemical properties of the ligand are optimally matched to the respective catalysis problem. Variation of the substituents in such compounds allows the electronic and steric properties of the ligand to be influenced in a targeted way, so that selectivity and activity in homogeneously catalyzed processes can be controlled. There is thus a great need for chiral ligands that are stereochemically and electronically different so as to provide optimally tailored ligands for a particular asymmetric catalysis reaction.

The structural variety of oxygen/nitrogen ligands (N/O ligands) known at the present time is very wide. These ligands can be classified, for example, according to the chemical functionality of the ligands. Examples of such classes of ligands are amino alcohols, imino alcohols, amino ethers, etc. This classification according to chemical functionality is particularly useful for describing the electronic properties of the ligands.

In addition, nitrogen/oxygen ligands can also be classified according to their symmetry properties or according to the number of coordination sites occupied by the ligands. This classification reflects, in particular, the stability, activity and stereoselectivity of metal complexes with oxygen/nitrogen ligands as catalyst precursors or as catalysts. Apart from the widespread C₂-symmetric ligand systems such as Salen (E. N. Jacobsen et al., J. Am. Chem. Soc., 1990, 112, 2801), asymmetric N/O ligands are becoming increasingly important in asymmetric catalysis. Important examples are the large class of chiral N/O ligands such as (1R,2S)-(+)-cis-1-amino-2-indanol (M. Wills et al., J. Org. Chem., 1997, 62, 5226), Schiff base ligands (A. H. Vetter and A. Berkessel, Tetrahedron Lett., 1998, 39, 1741), cis-2-amino-1-acenaphthenol (A. Sudo, M. Matsumoto, Y. Hashimoto, K. Saigo, Tetrahedron Asymmetry, 1995, 6 (8), 1853) and 1-(2-methoxy-1-naphthyl)isoquinoline (G. Chelucci et al., Tetrahedron Lett., 1999, 40, 553). The structures of these ligands are shown below.

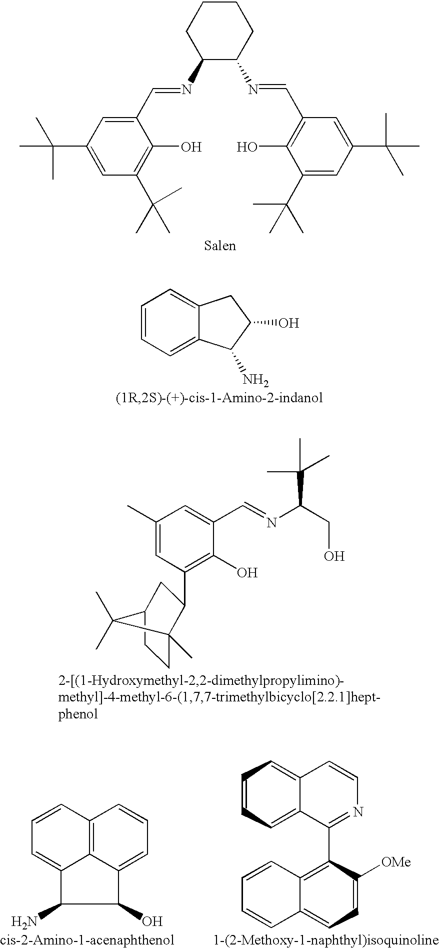

An important aspect of these classes of compounds is the creation of a highly asymmetric environment around the metal center due to coordination of the metal with these ligand systems. To utilize such an environment for effective transfer of the chirality, it is advantageous to control the flexibility of the ligand system in order to control the asymmetric induction.

### SUMMARY OF THE INVENTION

It is therefore an object of the present invention to provide novel, asymmetric, polydentate chiral nitrogen/oxygen ligands (N/O ligands) whose steric and electronic properties can readily be varied over a wide range. A second object of the present invention is to provide novel complexes of the asymmetric, polydentate ligands of the present invention with at least one metal atom or ion. A third object of the present invention is a method of catalyzing asymmetric reactions with the complexes of the present invention. A fourth object of the present invention is a method of preparing the asymmetric, polydentate ligands of the present invention.

### DETAILED DESCRIPTION OF THE INVENTION

The first object of the present invention is achieved by providing a class of asymmetric polydentate N/O ligands of the formulae (I) and (II) described below. The present invention accordingly provides compounds of the formulae (I) and (II),

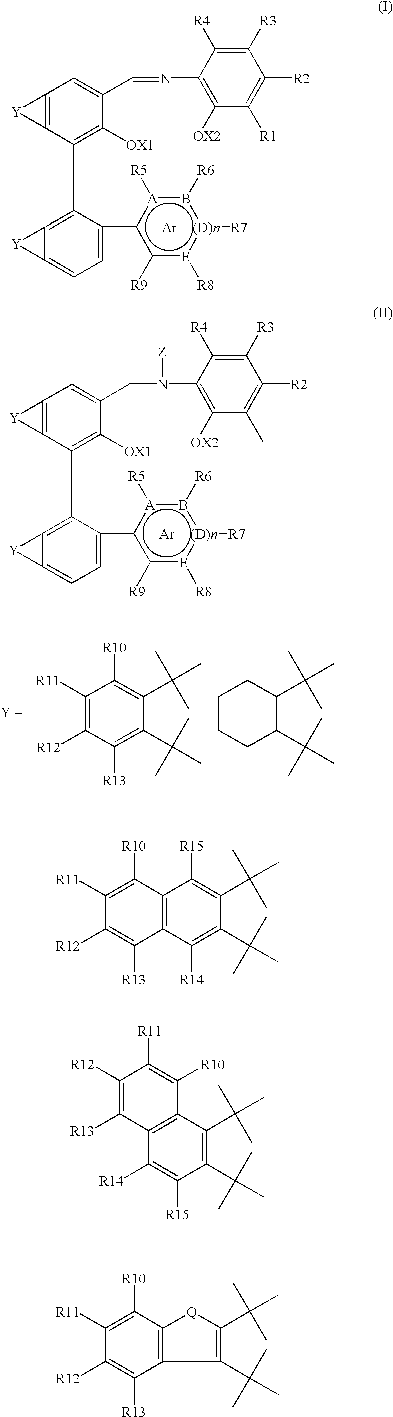

where n can be 0 or 1 and

Ar is part of a 6-membered aromatic or 5-6-membered heteroaromatic ring system, where the heteroaromatic ring system can have either 1-3 nitrogen atoms or 1 oxygen atom or 1 sulfur atom in the positions A, B, D, E and a six-membered heteroaromatic is preferably a pyridyl radical and a five-membered heteroaromatic is preferably a furyl, thienyl or pyrrolyl radical,

R1-R9 may each be, independently of one another, a hydrogen atom or C₁-C₂₀-alkyl, C₁-C₁₀-haloalkyl, C₃-C₈-cycloalkyl, C₅-C₈-cycloalkenyl, where the ring may also contain 1-2 heteroatoms selected from the group consisting of N, O and S, C₆-C₁₄-aryl, phenyl, naphthyl, fluorenyl, C₂-C₁₃-heteroaryl, where the number of heteroatoms selected from the group consisting of N, O and S can be 1-4, where the cyclic aliphatic or aromatic radicals are preferably 5- to 7-membered rings, where the abovementioned groups may in turn have one or more substituents, where the substituents may each be, independently of one another, hydrogen, C₁-C₂₀-alkyl, C₂-C₂₀-alkenyl, C₁-C₁₀-haloalkyl, C₃-C8-cycloalkyl, C₃-C₈-cycloalkenyl, C₂-C₉-heteroalkyl, C₁-C₉-heteroalkenyl, C₆-C₈-aryl, phenyl, naphthyl, fluorenyl, C₂-C₆-heteroaryl, where the number of hetero atoms from the group consisting of N, O and S can be 1-4, C₁-C₁₀-alkoxy, C₁-C₉-trihalomethylalkyl, trifluoromethyl, trichloromethyl, fluoro, chloro, bromo, iodo, cyano, C₁-C₈-alkyl or C₆-aryl, or may be tri-(C₁-C₆)-alkylsilyl, and where two of the substituents may be bridged, preferably bridged in such a way to provide a 5-7-membered aromatic or cycloaliphatic radical,

Z may be hydrogen, C₁-C₂₄-alkyl, benzyl, C₆-C₈-aryl, phenyl, naphthyl, allyl or vinyl,

X1, X2 may each be, independently of one another, hydrogen, methyl, ethyl, methoxymethyl, methoxyethyl,

Q may be oxygen, sulfur or nitrogen,

R10-R15 may each be, independently of one another, a hydrogen atom or C₁-C₂₀-alkyl, C₁-C₁₀-haloalkyl, C₃-C₈-cycloalkyl, C₅-C8-cycloalkenyl, where the ring may also contain 1-2 heteroatoms selected from the group consisting of N, O and S, C₆-C₁₄-aryl, phenyl, naphthyl, fluorenyl, C₂-C₁₃-heteroaryl, where the number of heteroatoms selected from the group consisting of N, O and S can be 1-4, where the cyclic aliphatic or aromatic radicals are preferably 5- to 7-membered rings, where the abovementioned groups may themselves each have one or more substituents, where these substituents may each be, independently of one another, hydrogen, C₁-C₂₀-alkyl, C₂-C₂₀-alkynyl, C₂-C₂₀-alkenyl, C₁-C₁₀-haloalkyl, C₃-C₈-cycloalkyl, C₃-C₈-cycloalkenyl, C₂-C₉-heteroalkyl, C₁-C₉-heteroalkenyl, C₆-C₈-aryl, phenyl, naphthyl, fluorenyl, C₂-C₆-heteroaryl, where the number of heteroatoms from the group consisting of N, O and S can be 1-4, C₁-C₁₀-alkoxy, C₁-C₉-trihalomethylalkyl, trifluoromethyl, trichloromethyl, fluoro, chloro, bromo, iodo, cyano, carboxylato of the formulae COOH and COOM′ where M′ is either a monovalent cation or C₁-C₄-alkyl, or C₁-C₆-acyloxy, sulfinato, C₁-C₈-alkyl or C₆-aryl, or can be tri-(C₁-C₆)-alkylsilyl.

The invention also provides complexes comprising at least one such chiral ligand system of the formula (I) or (II), together with at least one metal atom or ion.

A particular advantage is the modular structure of these ligand systems, which allows a wide range of steric and electronic variations to be provided by means of structural modifications to the ligands that may be easily introduced. As a result of this wide range of possible variations, in each case the ligand may be optimized for a specific catalysis problem in order to increase the selectivity and activity of the catalyst. A further advantage of the ligands of the present invention is that they can create a highly asymmetric coordination sphere in a metal complex.

Preferably the groups R1-R15 of the ligands of the present invention may be, independently, in addition to hydrogen, alkyl, alkenyl, cycloalkyl, alkoxy or trialkylsilyl substituents, preferably having up to 10, particularly preferably up to 6, carbon atoms. The following alkyl and alkoxy substituents are particularly preferred: methyl, ethyl, n-propyl, 1-methylethyl, n-butyl, 1-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, n-heptyl, n-octyl, n-nonyl, n-decyl, methoxy, ethoxy, 1-propoxy, 2-propoxy, 1-butoxy, 2-butoxy, 1,1-dimethylethoxy.

Substituted and unsubstituted cyclopentyl, cyclohexyl and cycloheptyl radicals are particularly preferred cyclic alkyl substituents. Particularly preferred alkenyl radicals are, for example, vinyl, propenyl, isopropenyl, 1-butenyl, 2-butenyl, 1-pentenyl, 2-pentenyl, 2-methyl -1-butenyl, 2-methyl-2-butenyl, 3-methyl-1-butenyl, 1-hexenyl, 1-heptenyl, 2-heptenyl, 1-octenyl or 2-octenyl groups.

Cyclopentenyl, cyclohexenyl, cycloheptenyl and norbornyl groups are particularly preferred cyclic alkenyl substituents.

Particularly preferred aryl substituents for R1-R15, are, for example, phenyl, 2-methylphenyl, 3,5-dimethylphenyl, 4-methylphenyl, 4-methoxyphenyl, 3,5-bis-(trifluoromethyl)phenyl, 4-trifluoromethylphenyl, 3,5-dimethyl-4-methoxyphenyl, 4-dialkylaminophenyl, 2-alkylphenyl, 3-alkylphenyl, 4-alkylphenyl, 2,6-dialkylphenyl, 3,5-dialkylphenyl, 3,4,5-trialkylphenyl, 2-alkoxyphenyl, 3-alkoxyphenyl, 4-alkoxyphenyl, 2,6-dialkoxyphenyl, 3,5-dialkoxyphenyl, 3,4,5-trialkoxyphenyl, 3,5-dialkyl-4-alkoxyphenyl, where the abovementioned alkyl and alkoxy groups each preferably contain from 1 to 6 carbon atoms.

All haloalkyl groups preferably have one of the formulae CHal₃, CH₂CHal₃, C₂Hal₅, where Hal may be, in particular, F, Cl or Br. Haloalkyl groups having the formulae CF₃, CH₂CF₃, C₂F₅ are particularly preferred.

Finally, particularly preferred ligand systems of the formulae (I) and (II) are optically active ligand systems which are enriched in one enantiomer, especially ligand systems in which the enantiomeric enrichment exceeds 90%, in particular 98%.

The class of polydentate N/O ligands of the present invention has a readily and variously modifiable chiral ligand skeleton whose steric and electronic properties can be varied over a very wide range due to a modular structure and the ready ability to introduce the most varied substituents. N/O ligands of the formulae (I) and (II) are able to create a highly asymmetric coordination sphere around the metal center in organometallic complexes and thus make possible effective asymmetric induction. In addition, the ability to easily introduce the most varied substituents into the N/O ligands enables the flexibility of the coordination sphere of the complex to be sterically controlled.

A wide range of applications can therefore be developed for compounds of the formulae (I) and (II), since the polydentate N/O ligands can be optimized sterically and electronically by a catalytic synthetic method for introducing appropriate substituents. At the same time, the compounds of the invention may be distinguished from many established ligand systems, because a wide range of variations may be synthesized in a particularly simple way starting from commercially available starting materials. This makes it easy to prepare the ligands of the present invention on an industrial scale. The choice of an appropriate method of preparation depends on the availability of the corresponding starting materials and on the desired substitution pattern. As is shown below, methods for synthesizing of the ligands of the present invention may be described by means of simple examples, without being restricted thereto.

N/O ligands according to the invention can be prepared as follows: The addition of a base and a sulfonation reagent to a binaphthol derivative in a suitable solvent provides a first coupling intermediate, which is coupled with another coupling intermediate in a coupling reaction catalyzed by a transition metal. An ortho-metallation, followed by a carbonylation reaction, gives the corresponding carbonyl intermediate, which is subsequently reacted with an amine component to form the imine (I) of the invention or is converted by direct reductive amination into the amine (II). The amine (II) can also be obtained by reduction of the corresponding imine (I). The process just described is illustrated below by means of preferred embodiments. 2-Aryl-2′-hydroxy-1,1′-binaphthyl is prepared in two steps by a method based on that of H. Sasaki, R. Irie, T. Hamada, K. Suzuki and T. Katsuki, Tetrahedron, 1994, 50(41), 11827-38.

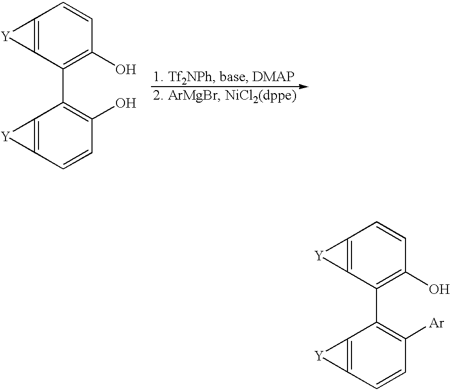

As an alternative, the compound is also obtainable via a Suzuki coupling reaction (as described by B. Schilling and D. E. Kaufmann, Eur. J. Org. Chem., 1998, 4, 701-9).

The carbonyl group is preferably introduced via an ortho-metallation. In this method, the hydroxy group is first converted into an ortho-directing group, lithiated by means of a lithium base and carbonylated by means of dimethylformamide. After removal of the ortho-directing group, a β-hydroxyaldehyde is obtained.

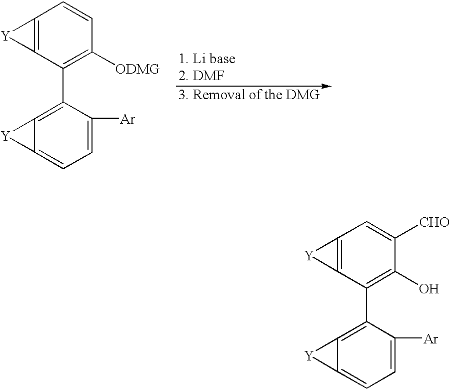

The aldehyde may be reacted with a wide variety of aromatic 2 hydroxyamines to form the imines (I) of the invention. The amines (II) can be obtained either directly by reductive amination of the β-hydroxyaldehydes or by reduction of the imines (I).

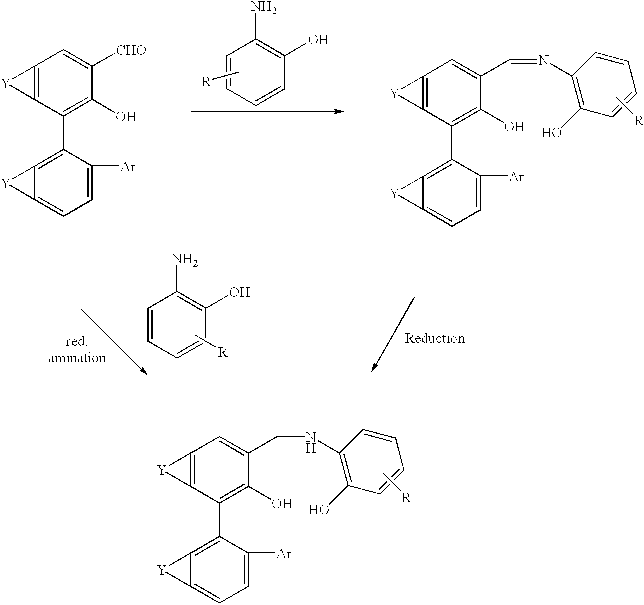

Accordingly, the present invention further provides a process for preparing ligands of the formulae (I) and (II), which comprises the following process steps:

a) Sulfonation of a 2,2′-dihydroxy-1,1′-biaromatic of the formula

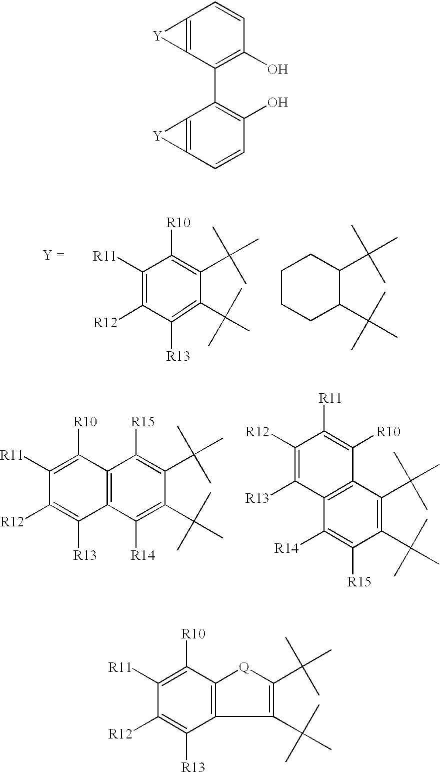

in the presence of a base, b) coupling of the sulfonated product obtained in step a) with an ArMgBr compound, where

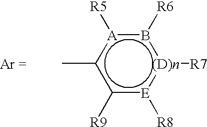

in the presence of a transition metal catalyst, c) protection of the free hydroxy groups, d) carbonylation of the product obtained under c) in the ortho position relative to the protected hydroxy group, e) removal of the protective group from the hydroxy function and reaction of the carbonylated product obtained in step d) with an aromatic amine of the formula

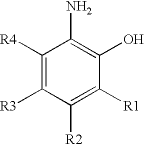

The compounds of the formulae (I) and (II) can be used as ligands for metals in metal-catalyzed reactions (e.g. reduction with inorganic hydrides, transfer hydrogenations, hydrosilylation, opening of epoxides, epoxidations, allylic oxidations, Baeyer-Villiger oxidations, oxidation of sulfides, cyclopropanations, Diels-Alder reactions, Michael additions, cyanohydrin reactions, Strecker and Strecker-type reactions, ene reactions, [3+2]-cycloadditions, Grignard reactions, addition of organozinc compounds onto carbonyl compounds or aldol reactions). They are particularly useful for asymmetric reactions. In particular, metal complexes of the ligands of the present invention are advantageous in asymmetric reactions in that the ligands of formulae (I) and (II) can be very well matched sterically and electronically to the respective substrate and the catalytic reaction due to the ease with which they can be modified in a wide variety of ways.

The complexes of the present invention comprise at least one ligand of formulae (I) and (II) complexed with at least one metal atom or ion, in particular aluminum, zinc, magnesium, titanium, zirconium, iron, nickel, cobalt, chromium, boron, copper, platinum, palladium, osmium, iridium, scandium, cerium, tin, manganese, rhodium or ruthenium. It is preferable that the complexes of the present invention have less than four metal centers, and it is particularly preferred that the complexes of the present invention have one or two metal centers. The metal centers may be occupied by different metal atoms and/or ions.

These metal-ligand complexes may be prepared in situ by the reaction of a metal compound or a metal salt or an appropriate precursor complex with the ligands of the formulae (I) and (II). Furthermore, a metal-ligand complex may be obtained by the reaction of a metal compound or a metal salt or an appropriate precursor complex with the ligands of the formulae (I) and (II), followed by subsequent isolation steps.

The complexes based on one or more metals, preferably metallic elements selected from the group consisting of Ru, Co, Rh, Ir, Ni, Pd, Pt, Cu, Al, Zn, Mn, Fe, Sc, Ti, Zr, Sn, B, Os, Ce, and at least one novel ligand of the formula (I) or (II), can themselves be used as a catalyst. However, the complexes can also be used as starting material for preparing modified catalysts.

Some illustrative embodiments of the invention are described below:

General Procedure

Reactions of air-sensitive compounds were carried out in an argon-filled glove box or in standard Schlenk tubes. Tetrahydrofuran (THF), diethyl ether and dichloromethane solvents were degassed and dried by means of a solvent drying unit (Innovative Technologies) by filtration through a column of activated aluminum oxide. Toluene and pentane were additionally freed of oxygen by means of a column filled with a copper catalyst.

The following examples serve to illustrate the invention, but do not restrict it in any way.

### EXAMPLE 1

### (R)-2-Hydroxy-2′-trifluoromethanesulfony-1,1′-binaphthyl

Under argon, 1.631 ml (14 mmol) of lutidine and 170.9 mg (1.4 mmol) of DMAP were added to a solution of 3.817 g (13.33 mmol) of (R)-2,2′-dihydroxy-1,1′-binaphthyl in 50 ml of absolute dichloromethane. The mixture was cooled to 0° C. and mixed with 5.00 g (14 mmol) of N,N-bis(trifluoromethanesulfonyl)aniline. After the addition was complete, the mixture was slowly warmed to room temperature and, after 3 days, evaporated to dryness. Purification was carried out by flash column chromatography on silica gel with a toluene eluant. The crude product was reacted further without additional characterization.

Yield. 7.12 g (crude product)

### EXAMPLE 2

### (R)-2-Hydroxy-2′-phenyl-1,1′-binaphthyl

Under argon, 138.2 mg (0.26 mmol) of (dppe)nickel(II) chloride and 17.4 ml (52.12 mmol) of phenyl magnesium bromide (3 molar solution in dichloromethane) were added to a solution of 7.12 g of (R)-2-hydroxy-2′-trifluoromethanesulfonyl-1,1′-binaphthyl in 80 ml of absolute diethyl ether. The mixture was stirred for 1 hour under reflux and overnight at room temperature and the reaction was stopped by addition of saturated ammonium chloride solution. The resulting mixture was extracted with diethyl ether, the organic phases were washed with sodium hydrogen carbonate solution and with water, dried over magnesium sulfate, filtered and evaporated under reduced pressure. Purification was carried out by flash column chromatography on silica gel with an isohexane/ethyl acetate (10:1) eluant.

Yield: 4.51 g (brown oil)

¹H-NMR (CDCl₃):

δ=8.00-6.90 (m, 17H, aromatic), 5.10 (s, 1H, OH)

### EXAMPLE 3

### (R)-2-Methoxy-2′-phenyl- 1,1′-binaphthyl

Under argon, 7.67 ml (43.44 mmol) of N,N-diisopropylethylamine and 3.33 ml (43.44 mmol) of chloromethyl methyl ether were added to a solution of 4.51 g of (R)-2-hydroxy -2′-phenyl-1,1′-binaphthyl in 60 ml of absolute dichloromethane. After 24 hours, the reaction was stopped by addition of 10 ml of water. The mixture was extracted with dichloromethane, the organic phases were washed with water and dried over magnesium sulfate. The magnesium sulfate was filtered off and the filtrate was evaporated under reduced pressure. The product was reacted further without additional purification.

### EXAMPLE 4

### (R)-3 -Formyl-2-methoxy-2′-phenyl-1,1′-binaphthyl

Under argon, 17.92 ml (26.06 mmol) of n-butyllithium (1.6 molar solution in hexane) was added at −78° C. to a solution of 13.03 mmol of (R)-2-methoxy-2′-phenyl-1,1′-binaphthyl in 55 ml of absolute tetrahydrofuran. After 3 hours, the reaction mixture was mixed with 5.07 ml (65.15 mmol) of dimethylformamide and stirred while thawing overnight. The reaction was stopped by addition of ammonium chloride solution and the mixture was extracted with dichloromethane. The organic phases were washed with sodium hydrogen carbonate solution and with water, dried over magnesium sulfate, filtered and evaporated under reduced pressure. The product was reacted further without additional purification.

### EXAMPLE 5

### (R)-3-Formyl-2-hydroxy-2′-phenyl-1,1′-binaphthyl

Under argon, 2.0 g of 4 Å molecular sieves and 6.87 ml (4 eq., 52.82 mmol) of bromotrimethylsilane were added to a solution of the crude product from Example 4 in 50 ml of absolute dichloromethane. After 3 hours, the reaction mixture was mixed with saturated sodium hydrogen carbonate solution, resulting in foaming, and extracted with dichloromethane. The organic phases were dried over magnesium sulfate, filtered and evaporated under reduced pressure. Purification was carried out by flash column chromatography on silica gel with an isohexane/toluene (3:7) eluant, and then a second flash column chromatography step with a second silica gel column, using an isohexane/ethyl acetate (10:1) eluent. Evaporation resulted in crystallization of the compound in the form of yellow crystals.

Yield: 718 mg of yellow crystals (2.07 mmol, 15.9% over 5 steps)

¹H-NMR (CDCl₃):

δ=10.40 (s, 1H, CHO), 10.10 (s, 1H, OH), 8.20-6.90 (m, 16 H, aromatic)

### EXAMPLE 6

### (R)-3-[(2-Hydroxyphenylimino)methyl]-2-hydroxy-2′-phenyl-1,1′-binaphthyl

Under argon, 187 mg (0.5 mmol) of(R)-3-formyl-2-hydroxy-2′-phenyl-1,1′-binaphthyl in 6 ml of absolute dichloromethane was added to 500 mg of heat-dried molecular sieves, and 57.3 mg (0.525 mmol) of 2-hydroxyaniline was then added. After 5 days, the mixture was filtered through heat-dried cellulose, eluted with 2 ml of absolute dichloromethane and then the solvent was removed under reduced pressure.

Yield: light-brown solid (quantitative)

¹H-NMR (abs. CD₂Cl₂):

δ=12.26 (s, 1H, OH), 8.71 (s, 1H, imine-H), 8.05-6.83 (m, 20 H, aromatic)

¹³C-NMR (abs. CD₂Cl₂) 162.16 (imine-C)

Catalysis Experiments

### EXAMPLE 7

### Addition of diethylzinc onto benzaldehyde

Under argon, 0.021 mmol of (R)-3-[(2-hydroxyphenylimino)methyl]-2-hydroxy-2′-phenyl -1,1′-binaphthyl was dissolved in 10 ml of absolute toluene. 115 μl of benzaldehyde was added to this solution at room temperature and the mixture was subsequently stirred for 20 minutes. The solution was then cooled to −30° C. and then 1.5 ml of 1M diethylzinc solution was added dropwise. The solution was warmed to −5° C. over a period of 60 minutes and stirred at this temperature for another 24 hours. The reaction solution was hydrolyzed using 10 ml of 1M hydrochloric acid and the aqueous phase was extracted three times with 20 ml each time of dichloromethane. After the organic phases were dried, the solvent was removed and the crude product was purified by means of chromatography. The enantiomeric excess (ee) was determined by means of chiral HPLC (Chiralcel OD).

Yield: 85%
ee: 70%

### EXAMPLE 8

### 1,4-Addition onto 2-cyclohexenone

Under argon, 0.03 mmol of (R)-3-[(2-hydroxyphenylimino)methyl]-2-hydroxy-2′-phenyl -1,1′-binaphthyl and 0.025 mmol of copper(II) triflate were dissolved in 5 ml of absolute acetonitrile and the mixture was stirred at room temperature for 1 hour. The solution was subsequently cooled to −30° C. 100 mg of 2-cyclohexenone and 2.2 ml of 1M diethylzinc solution were added to this solution. The reaction solution was stirred at −30° C. for 24 hours, subsequently warmed to room temperature, diluted with 10 ml of diethyl ether and hydrolyzed using 10 ml of ammonium chloride solution. The aqueous phase was extracted three times with 25 ml of diethyl ether. After drying, the solvent was removed and the crude product was purified by means of chromatography. The enantiomeric excess was determined by means of chiral gas chromatography (Lipodex A).

Yield: 79%
ee: 57%

The priority document of the present application, German application 101 22 539.3, filed May 9, 2001, is incorporated herein by reference.

Obviously, numerous modifications and variations on the present invention are possible in light of the above teachings. It is therefore to be understood that within the scope of the appended claims, the invention may be practiced otherwise than as specifically described herein.

What is claimed as new and is intended to be secured by Letters Patent is:

1. A chiral, asymmetric, polydentate nitrogen/oxygen ligand of the formula (I) or (II), 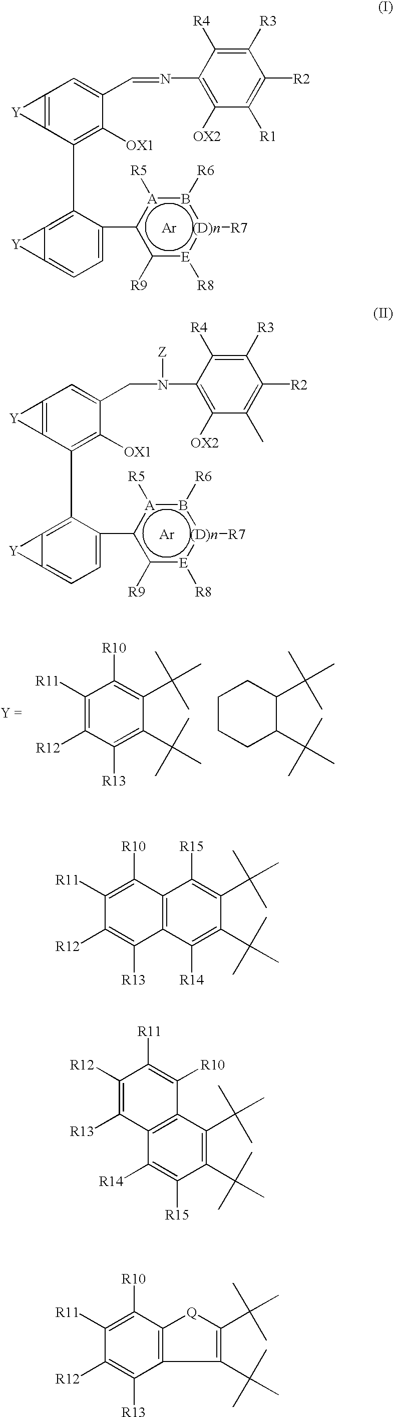
where n can be 0 or 1 and
Ar defines a 6-membered aromatic or 5-6-membered heteroaromatic ring system, where the heteroaromatic ring system can have either 1-3 nitrogen atoms or 1 oxygen atom or 1 sulfur atom in the positions A, B, D, E, and
R1-R9 are each, independently of one another, selected from the group consisting of a hydrogen atom, C₁-C₂₀-alkyl, C₁-C₁₀-haloalkyl, C₃-C₈-cycloalkyl, C₅-C₈-cycloalkenyl, where the ring may also contain 1-2 heteroatoms selected from the group consisting of N, O and S, C₆-C₁₄-aryl, phenyl, naphthyl, fluorenyl, and C₂-C₁₃-heteroaryl, where the number of heteroatoms selected from the group consisting of N, O and S can be 1-4, and
the abovementioned groups may in turn have one or more substituents, where the substituents are each, independently of one another, selected from the group consisting of hydrogen, C₁-C₂₀-alkyl, C₂-C₂₀-alkenyl, C₁-C₁₀-haloalkyl, C₃-C₈-cycloalkyl, C₃-C₈-cycloalkenyl, C₂-C₉-heteroalkyl, C₁-C₉-heteroalkenyl, C₆-C₈-aryl, phenyl, naphthyl, fluorenyl, C₂-C₆-heteroaryl, where the number of hetero atoms selected from the group consisting of N, O and S can be 1-4, C₁-C₁₀-alkoxy, C₁-C₉-trihalomethylalkyl, trifluoromethyl, trichloromethyl, fluoro, chloro, bromo, iodo, cyano, C₁-C₈-alkyl, C₆-aryl, and tri-(C₁-C₆)-alkylsilyl, where two of the substituents can also be bridged, and
Z is selected from the group consisting of hydrogen, C₁-C₂₄-alkyl, benzyl, C₆-C₈-aryl, phenyl, naphthyl, allyl and vinyl, and
X1, X2 are each, independently of one another, selected from the group consisting of hydrogen, methyl, ethyl, methoxymethyl, and methoxyethyl, and
Q is selected from the group consisting of oxygen, sulfur and nitrogen, and
R10-R15 are each, independently of one another, selected from the group consisting of a hydrogen atom, C₁-C₂₀-alkyl, C₁-C₁₀-haloalkyl, C₃-C₈-cycloalkyl, C₅-C₈-cycloalkenyl, where the ring may also contain 1-2 heteroatoms selected from the group consisting of N, O and S, C₆-C₁₄-aryl, phenyl, naphthyl, fluorenyl, and C₂-C₁₃-heteroaryl, where the number of heteroatoms selected from the group consisting of N, O and S can be 1-4,
where the abovementioned groups may themselves each bear one or more substituents, where these substituents are each, independently of one another, selected from the group consisting of hydrogen, C₁-C₂₀-alkyl, C₂-C₂₀-alkynyl, C₂-C₂₀-alkenyl, C₁-C₁₀-haloalkyl, C₃-C₈-cycloalkyl, C₃-C₈-cycloalkenyl, C₂-C₉-heteroalkyl, C₁-C₉-heteroalkenyl, C₆-C₈-aryl, phenyl, naphthyl, fluorenyl, C₂-C₆-heteroaryl, where the number of heteroatoms from the group consisting of N, O and S can be 1-4, C₁-C₁₀-alkoxy, C₁-C₉-trihalomethylalkyl, trifluoromethyl, trichloromethyl, fluoro, chloro, bromo, iodo, cyano, carboxylato of the formulae COOH and COOM′ where M′ is either a monovalent cation, C₁-C₄-alkyl, C₁-C₆-acyloxy, sulfinato, C₁-C₈-alkyl, C₆-aryl, and tri-(C₁-C₆)-alkylsilyl.

2. The ligand of claim 1, wherein X1 and X2 are each hydrogen.

3. The ligand of claim 1, wherein Ar is a pyridyl, furyl, thienyl or pyrrolyl radical.

4. The ligand of claim 1 which is enantiomerically enriched.

5. The ligand of claim 1, wherein the enantiomeric enrichment exceeds 90%.

6. The ligand of claim 1, wherein the enantiomeric enrichment exceeds 98%.

7. A complex comprising at least one ligand as claimed in claim 1 and at least one metal atom or ion.

8. A complex prepared by reacting at least one metal, metal salt or metal precursor complex with at least one ligand as claimed in claim 1.

9. The complex of claim 7, wherein said metal atom or ion is at least one metal atom or ion selected from the group consisting of aluminum, zinc, magnesium, titanium, zirconium, iron, nickel, cobalt, chromium, boron, copper, platinum, palladium, osmium, iridium, scandium, cerium, tin, manganese, rhodium, ruthenium and a mixture thereof.

10. A method of preparing an asymmetric molecule comprising catalytically reacting at least one starting material with a complex according to claim 7.

11. The method as claimed in claim 10, wherein said catalytically reacting is selected from the group consisting of an asymmetric transfer hydrogenation, a reduction with an inorganic hydride, a hydrosilylation, an epoxide ring opening, an epoxidation, an allylic oxidation, a Baeyer-Villiger oxidation, an oxidation of a sulfide, a cyclopropanation, a Diels-Alder reaction, a Michael addition, a cyanohydrin reaction, a Strecker or Strecker-type reaction, an ene reaction, a [3+2] cycloaddition, a Grignard reaction, an addition of an organozinc compound onto a carbonyl compound, and an aldol reaction.

12. A process for preparing the ligands as claimed in claim 1, which comprises the following process steps:
a) Sulfonation of a 2,2′-dihydroxy-1,1′-biaromatic of the formula 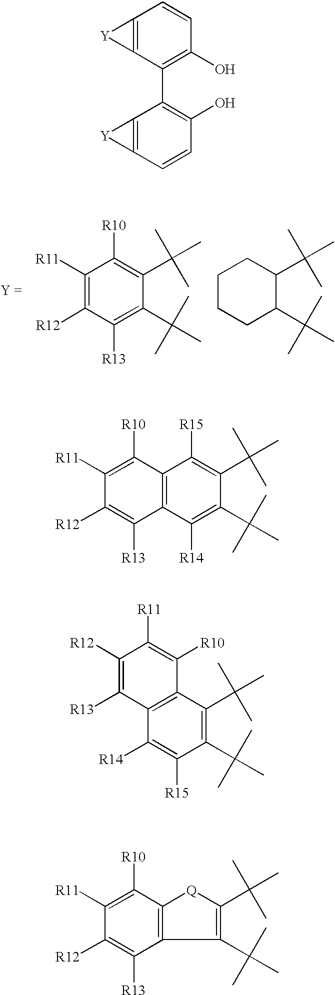
in the presence of a base,
b) Coupling of the sulfonated product from step a) with an ArMgBr compound, where 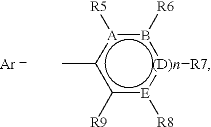
in the presence of a transition metal catalyst,
c) Protection of the free hydroxy groups,
d) Carbonylation of the product obtained from step c) in the ortho position relative to the protected hydroxy group,
e) Removal of the protective group from the hydroxy function and reaction of the carbonylated product from step d) with an aromatic amine of the formula

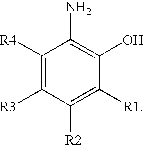

13. The process as claimed in claim 12, wherein the reaction of step e) is a reductive amination.

14. The process as claimed in claim 12, wherein the reaction of step e) is an amination with subsequent reduction.

15. The ligand as claimed in claim 1, wherein R1 to R15 are substituents selected from the group consisting of methyl, ethyl, n-propyl, 1-methylethyl, n-butyl, 1-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, n-heptyl, n-octyl, n-nonyl, n-decyl, methoxy, ethoxy, 1-propoxy, 2-propoxy, 1-butoxy, 2-butoxy, and 1,1-dimethylethoxy radicals.

16. The ligand as claimed in claim 1, wherein R1 to R15 are substituents selected from the group consisting of cyclopentyl, cyclohexyl and cycloheptyl radicals.

17. The ligand as claimed in claim 1, wherein R1 to R15 are substituents selected from the group consisting of vinyl, propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 1-heptenyl, 2-heptenyl, 1-octenyl and 2-octenyl radicals.

18. The ligand as claimed in claim 1, wherein R1 to R15 are substituents selected from the group consisting of cyclopentenyl, cyclohexenyl, cycloheptenyl and norbornyl radicals.

19. The ligand as claimed in claim 1, wherein R1 to R15 are substituents selected from the group consisting of phenyl, 2-methylphenyl, 3,5-dimethylphenyl, 4-methyl phenyl, 4-methoxyphenyl, 3,5-bis-(trifluoromethyl)phenyl, 4-trifluoromethylphenyl, 3,5-dimethyl-4-methoxyphenyl, 4-dialkylaminophenyl, 2-alkylphenyl, 3-alkylphenyl, 4-alkylphenyl, 2,6-dialkylphenyl, 3,5-dialkylphenyl, 3,4,5-trialkylphenyl, 2-alkoxyphenyl, 3-alkoxyphenyl, 4-alkoxyphenyl, 2,6-dialkoxyphenyl, 3,5-dialkoxyphenyl, 3,4,5-trialkoxyphenyl, 3,5-dialkyl-4-alkoxyphenyl
